(19) United States
(12) Patent Application Publication
(10) Pub. No.: US 2021/0341472 A1
(43) Pub. Date: Nov. 4, 2021

(54) SOLID-PHASE SURFACE AND SOLUTION MOTION MODE AND MOTION DEVICE

(71) Applicant: SHANGHAI JIAO TONG UNIVERSITY, Shanghai (CN)

(72) Inventors: Tian YANG, Shanghai (CN); Xiaolong HE, Shanghai (CN); Yalin WANG, Shanghai (CN)

(73) Assignee: Shanghai Jiao Tong University, Shanghai (CN)

(21) Appl. No.: 17/053,792

(22) PCT Filed: May 6, 2019
(86) PCT No.: PCT/CN2019/085622

§ 371 Date: Jul. 19, 2021

(30) Foreign Application Priority Data
May 9, 2018 (CN) 2018104383063
May 9, 2018 (CN) 2018206853266

(51) Int. Cl. G01N 33/543; G01N 21/552

(52) U.S. Cl. CPC G01N 33/54373; G01N 2201/0846; G01N 21/553

(57) ABSTRACT

The present disclosure provides modes of relative motion between a solid surface and a solution, and the related motion apparatuses. In an interaction between the solid surface and a target object, the target object is dissolved or dispersed in the solution, and the solid surface and the solution make a relative motion, with the relative motion including a relative movement perpendicular to the solid surface, in order to improve at least one of the following: binding rate, dissociation rate, binding uniformity, binding directionality and binding density of the target object to the solid surface. Compared with the traditional modes of relative motion in which the relative motion is parallel to a sensor surface, the modes of relative motion of the present disclosure can effectively improve the binding efficiency and dissociation efficiency between a ligand and an analyte given the same relative motion velocity between the sensor and the solution.

### TECHNICAL FIELD

The present disclosure belongs to the field of biosensing, and in particular, to the modes of relative motion between a solid sensor surface and a sample solution, and the related motion apparatuses.

### BACKGROUND

The measurement of the kinetic and thermodynamic processes of biomolecular interactions is an important cornerstone technology of life sciences and drug development. For example, the development of each new antibody drug uses hundreds of thousands of samples and goes through a series of steps including target screening, engineering cell line construction, clinical pharmacokinetics, immunogenicity testing and quality control, in which testing of biomolecular interactions is required in each step for making accurate evaluations. Among the key technologies for characterizing biomolecular interactions is label-free sensing, the application demands and industrial scale of which have been growing fast.

Compared with traditional measurement technologies such as enzyme-linked immunosorbent assay, the label-free sensing technology doesn't have to change the intrinsic characteristics of the samples (by eliminating the labeling process) and, at the same time, greatly saves operation time and labor. In addition, its unique real-time measurement capability has high values for scientific research and drug screening. At present, the most widely used label-free biosensing technology is based on the surface plasmon resonance (SPR) mechanism. In late 2016, SPR was included in the U.S. Pharmacopoeia and the Japan Pharmacopoeia, being an immunology test method for preclinical drug evaluation. SPR indicates resonance of a kind of surface waves resulting from coupling between free charge oscillation at a metallic surface and an electromagnetic field. The resonant coupling condition between SPR and incident light is very sensitive to the density of mass that adsorbs onto the metallic surface. When a layer of ligand molecules is immobilized on the metallic surface to specifically capture the target analyte molecules, the binding behaviors between the ligand molecules and the analyte molecules can be inferred according to the SPR optical signal, so that a number of parameters can be obtained accordingly including analyte concentration, specificity, affinity and kinetic constants.

To date, almost all of the commercial SPR products are based on microfluidics for sample injection, with the flagship products being Biacore's instruments and sensor chips. For this kind of sample injection method, the ligand is immobilized on the solid sensor chip, the analyte is in the sample solution, and the sample solution flows in the microfluidic channel along the direction parallel to the sensor surface at a speed up to the order of m/min. Such a microfluidic injection method keeps the sample solution constantly refreshed and also wipes the chip surface, which improves the analyte capturing efficiency and mitigates the effects of slow diffusion and other factors that hamper the binding and dissociation between the ligand and the analyte. Especially, for experiments with low analyte concentrations and/or slow analyte diffusion (mass transfer), the sample solution flow velocities need to be high to achieve efficient ligand-analyte binding and accurate interaction kinetics measurements.

Unlike the SPR sensing chips using microfluidic sample injection, in the Octet products of the company ForteBio, the flat end-facet of an optical cable serves as the solid sensor surface. The ligand is immobilized on this optical cable end-facet (the solid sensor surface), and the analyte is in a sample solution held by an open container such as a well of a micro-titre plate. While the SPR chips use high microfluid flow speeds to improve analyte capturing efficiencies, the sample solution container of Octet oscillates fast by circularly rotating in a plane which is parallel to the optical cable's end facet, with a rotation speed up to the order of 1000 rpm. Since the rotating speeds of the sample solution are different at different distances to the rotation center, the improvement of analyte capturing efficiency is not uniform. Therefore, the relative positioning between the sensor, the sample and the rotor will contribute to the errors of the experiment results. Moreover, the flow field distribution and the adsorbed molecules' distribution and orientation are different between under the unidirectional microfluid flow condition and under the rotating flow condition, which adds additional discrepancies to the experiment results.

The abovementioned two modes of relative motion between sensors and sample solutions have been widely employed in label-free biomolecular interaction experiments for more than ten or twenty years, respectively. They are, in short, 1) the sample solution flowing unidirectionally parallel to the solid sensor surface, and 2) the sample solution rotating around a center in a plane parallel to the solid sensor surface. Creating novel modes of motion for sensors and sample solutions that further increase the analyte capturing efficiencies is important for improving the sensitivities of biomolecular interaction experiments and the accuracies of kinetics measurement.

According to the above analysis, it is highly valuable to provide a novel mode of motion for solid sensor surfaces and analyte sample solutions that efficiently improves the binding and dissociation efficiencies between ligands and analytes.

### SUMMARY

The present disclosure provides a mode of relative motion between a solid surface and a solution, to solve the problems of unsatisfactory binding rate and dissociation rate between the solid surface and the target object in the solution.

The present disclosure provides a mode of relative motion between a solid surface and a solution. During an interaction between a solid surface and a target object, the target object is dissolved or dispersed in a solution, the solid surface and the solution make a relative motion, with the relative motion including a relative movement perpendicular to the solid surface, so as to improve at least one of the binding rate, dissociation rate, binding uniformity, binding directionality, and binding density of the target object to the solid surface.

Preferably, in the binding and dissociation processes between the solid surface and the target object, the relative motion between the solid surface and the solution includes a unidirectional opposite movement perpendicular to the solid surface, so as to improve at least one of the binding rate, dissociation rate, binding uniformity, binding directionality, and binding density of the target object to the solid surface.

Preferably, in the binding and dissociation processes between the solid surface and the target object, the relative motion between the solid surface and the solution includes reciprocating forward movement and backward movement perpendicular to the solid surface, so as to improve at least one of the binding rate, dissociation rate, binding uniformity, binding directionality, and binding density of the target object to the solid surface.

Preferably, the relative motion includes a combination of the relative movement perpendicular to the solid surface and the relative movement parallel to the solid surface.

Further, the relative movement perpendicular to the solid surface and the relative movement parallel to the solid surface are simultaneously made.

Further, the relative movement parallel to the solid surface includes one of a unidirectional movement, a bidirectional reciprocating movement, and a rotating movement.

Preferably, the relative motion between the solid surface and the solution includes one of following: only the solid surface moves, only the solution moves and both the solid surface and the solution move.

Preferably, a velocity of the perpendicular relative movement between the solid surface and the solution is not less than 1/10 of the Brownian motion velocity of the target object in the solution, in order to increase the probability of collision between the solid surface and the target object.

Preferably, the target object includes a large-particle analyte, and the large-particle analytes are selected from but not limited to a group consisting of metallic particles, exosome particles, cells, quantum dots, and dielectric particles, and a combination thereof.

Preferably, for a biomolecular interaction experiment, the solid surface includes a sensor surface with ligands immobilized thereon, and the target object includes analytes which are dissolved or dispersed in the solution; a binding and a dissociation between the target object and the solid surface refer to a binding and a dissociation between the analytes and the ligands, respectively.

Further, the sensor includes a sensor at an optical fiber end-facet or a sensor at an optical cable end-facet, the end-facet is approximately perpendicular to a lightwave propagation direction in a part of optical fiber or optical cable adjacent to the sensor, and the choice of sensor at an optical fiber end-facet includes a surface plasmon resonance (SPR) sensor and the choice of sensor at an optical cable end-facet includes a bio-layer interferometry sensor.

The present disclosure further provides a mode of relative motion between a solid surface and a solution. In an interaction between the solid surface and a target object, the target object is dissolved or dispersed in the solution, and the solid surface and the solution make a relative motion, with the relative motion including a reciprocating movement parallel to the solid surface other than rotation around a center, in order to improve the binding and dissociation rates between the target object and the solid surface.

Preferably, for a biomolecular interaction experiment, the solid surface includes a sensor surface with ligands immobilized thereon, and the target object includes analytes which are dissolved or dispersed in the solution; a binding and a dissociation between the target object and the solid surface refer to a binding and a dissociation between the analytes and the ligands, respectively. The sensor includes a sensor at an optical fiber end-facet or a sensor at an optical cable end-facet, the end-facet is approximately perpendicular to a lightwave propagation direction in a part of optical fiber or optical cable adjacent to the sensor, and the choice of sensor at an optical fiber end-facet includes a surface plasmon resonance (SPR) sensor and the choice of sensor at an optical cable end-facet includes a bio-layer interferometry sensor.

The present disclosure further provides a motion apparatus for a solid surface and a solution, including: a container, which holds a solution, with a target object dissolved or dispersed in the solution; a solid surface, which includes a sensor surface with ligands immobilized thereon for measuring biomolecular interactions, the sensor being immersed in the solution; and a drive motor, which is connected to the container to make a reciprocating motion of the container, the reciprocating motion including a movement perpendicular to the sensor surface, in order to improve at least one of the following: binding rate, dissociation rate, binding uniformity, binding directionality, and binding density of the target object to the sensor surface.

Preferably, the reciprocating motion of the container driven by the drive motor further includes a movement parallel to the sensor surface, so that the perpendicular and parallel movements of the container are simultaneously driven.

Preferably, the drive motor includes a voice coil motor.

Preferably, the container includes any one of a microtitre plate and a centrifuge tube.

Preferably, the sensor includes a sensor at an optical fiber end-facet or a sensor at an optical cable end-facet, the end-facet is approximately perpendicular to a lightwave propagation direction in a part of optical fiber or optical cable adjacent to the sensor, and the choice of sensor at an optical fiber end-facet includes a surface plasmon resonance (SPR) sensor and the choice of sensor at an optical cable end-facet includes a bio-layer interferometry sensor.

As described above, the modes of relative motion between a solid sensor surface and a sample solution and the related motion apparatuses of the present disclosure have the following beneficial effects:

1) The present disclosure provides a mode of relative motion in which the relative motion between the sensor surface and the solution is perpendicular to the solid surface. Compared with the traditional modes of relative motion in which the relative motion is parallel to a sensor surface, the mode of relative motion of the present disclosure can effectively improve the binding efficiency and dissociation efficiency between a ligand and an analyte given the same relative motion velocity between the sensor and the sample solution.

2) The perpendicular motion mode of the present disclosure will not cause significant nonuniformity of the flow rate of the sample solution, and it will effectively improve the binding uniformity, binding directionality and binding density of the analyte to the ligand.

3) The present disclosure can effectively improve the detection sensitivity for biomolecular interactions, and therefore shows promises for a broad range of applications in biosensing.

4) When the velocity of the perpendicular relative movement between the sensor and the sample solution is no less than 1/10 of the Brownian motion velocity of the analyte, such modes of relative motion will significantly increase the probability of ligand-analyte collision. For the measurement of analyte particles with large masses and low Brownian motion velocities, such as metallic particles, exosome particles, cells, quantum dots and dielectric particles, the present disclosure can remarkably increase the velocity of relative movement between the analyte and the ligand.

5) In the present disclosure, the sensor and sample solution conduct a reciprocating relative movement parallel to the sensor surface, which is different from rotation around a center. This mode of motion can also significantly improve the ligand-analyte binding efficiency compared to when there is no movement. In addition, the relative motion velocity under this motion mode does not vary with the spatial offset of the sensor from the center of the sample solution, so that the consistency between different testing results shall be improved compared with the rotating-around-a-center relative motion mode.

### BRIEF DESCRIPTION OF THE DRAWINGS

FIG. 1 is a schematic diagram of a sensing apparatus of the present disclosure.

FIGS. 2 to 4 are schematic diagrams showing several modes of relative motion between a solid surface and a solution according to the present disclosure.

FIG. 5 is a schematic diagram showing a mode of relative motion in which an optical fiber reciprocatively moves to the left and right along the horizontal direction.

FIG. 6 is a schematic diagram showing a mode of relative motion in which a microtitre plate circularly rotates around a center.

FIG. 7 shows the experiment results for PPB-SA molecular interaction according to the present disclosure.

FIGS. 8 to 10 show the experiment results for BPA-hIgG molecular interaction according to the present disclosure.

FIG. 11 is a schematic diagram of a motion apparatus for a solid surface and a solution according to the present disclosure.

### DESCRIPTION OF REFERENCE NUMERALS

| | |
|---|---|
| 10 | Light source |
| 11 | Directional coupler |
| 12 | Optical fiber |
| 13 | Sample solution |
| 14 | Sensor on optical fiber end-facet |
| 15 | Spectrometer |
| 16 | Analyte |
| 21 | Container |
| 22 | Drive motor |

### DETAILED DESCRIPTION OF THE PREFERRED EMBODIMENTS

The embodiments of the present disclosure will be described below. Those skilled in the art can easily understand other advantages and effects of the present disclosure according to contents disclosed by the specification. The present disclosure may also be implemented or applied through other different specific implementation modes. Various modifications or changes may be made to all details in the specification based on different points of view and applications without departing from the spirit of the present disclosure.

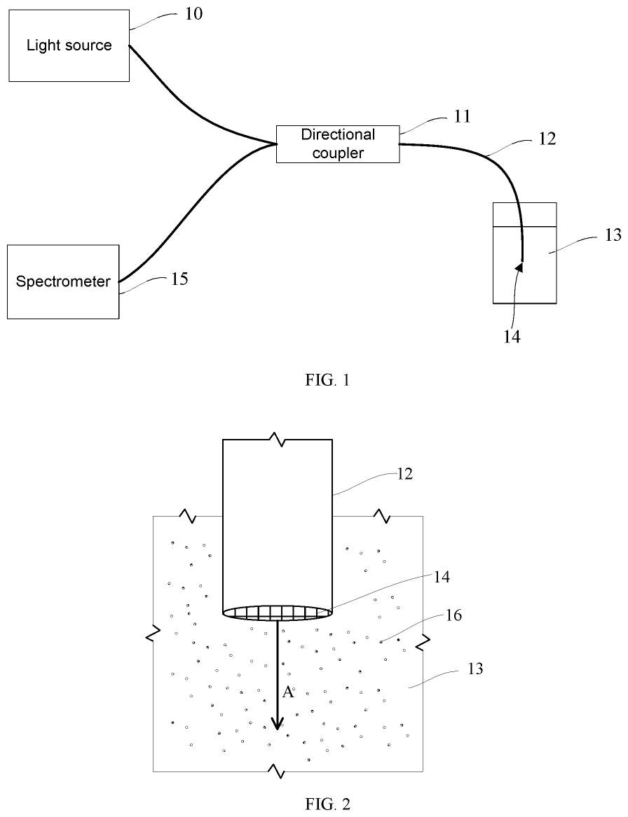

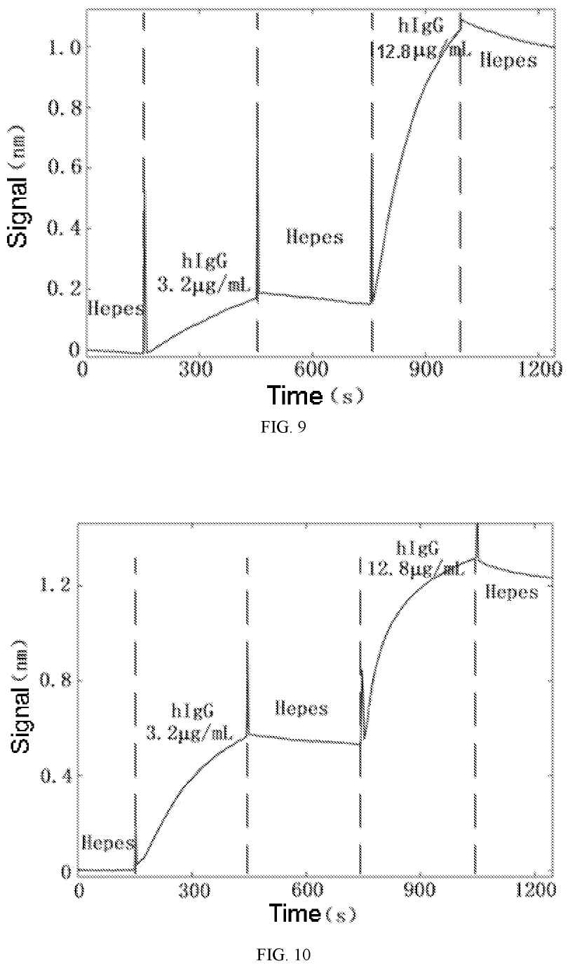

Referring to FIGS. 1-11. It needs to be stated that the drawings provided in the following embodiments are just used for schematically describing the basic concept of the present disclosure, thus only illustrating components only related to the present disclosure and are not drawn according to the numbers, shapes and sizes of components during actual implementation, the configuration, number and scale of each component during actual implementation thereof may be freely changed, and the component layout configuration thereof may be more complicated.

As shown in FIGS. 2 and 4, this embodiment provides a mode of relative motion between a solid surface and a solution. During interaction of a solid surface and a target object, the target object is dissolved or dispersed in a solution, the solid surface and the solution make a relative motion, with the relative motion including a relative movement perpendicular to the solid surface, so as to improve at least one of the binding rate, dissociation rate, binding uniformity, binding directionality, and binding density of the target object to the solid surface. For example, in the binding and dissociation processes between the solid surface and the target object, the relative motion between the solid surface and the solution may include a unidirectional opposite movement A perpendicular to the solid surface (as shown in FIG. 2) or include a reciprocating forward movement A and a backward movement B perpendicular to the solid surface, so as to improve binding rate, binding uniformity, binding directionality, and binding density of the target object to the solid surface. In the interaction process between the solid surface and the target object, the factors that interfere with the binding between the solid surface and the target object include: 1) there is a thin film of still water right on top of the solid surface which will slow down the binding and dissociation of the analyte; 2) the diffusion velocity of the analyte in the solution will limit its binding rate to the solid surface; 3) insufficient exposure of the sites on the ligand that could bind to the analyte will reduce the binding rate of the analyte. For the above reasons, it is usually necessary to use a relative motion between the solid surface and the target object to reduce the thickness of the still water film on the solid surface, to increase the diffusion rate of the analyte, and to help expose the binding sites of the ligand. Experiments have shown that, compared with the parallel motion modes and the rotational motion modes, the perpendicular motion mode of the solid surface and the target object according to the present disclosure produced more efficient molecular binding, which might be attributed to more efficient scouring of the solid surface in the perpendicular motion mode.

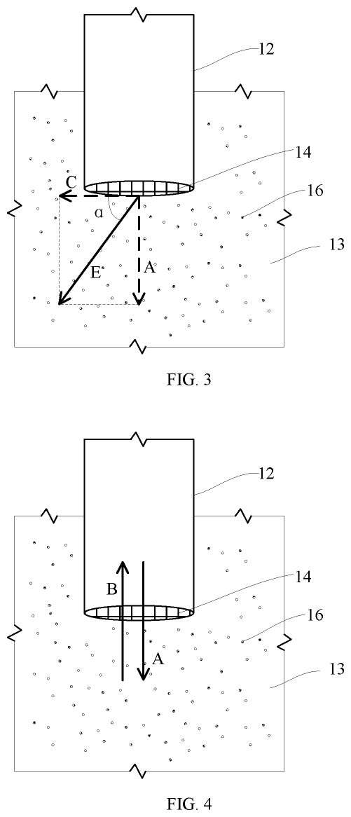

In addition, as shown in FIG. 3, in the binding and dissociation process between the solid surface and the target object, the relative motion between the solid surface and the solution may include a combination of the relative movement perpendicular to the solid surface and a relative movement C parallel to the solid surface. If the relative movement perpendicular to the solid surface (such as the forward movement A) and the relative movement C parallel to the solid surface are simultaneously made, the direction of the relative motion between the solid surface and the solution to be tested will be at an angle α to the solid surface, and the actual relative motion direction is shown as the direction E in FIG. 3. Furthermore, the relative movement parallel to the solid surface includes one of a unidirectional movement, a bidirectional reciprocating movement, and a rotational movement. According to actual needs, an appropriate combination of motion modes can further improve the scouring effect on the solid surface compared with a single perpendicular motion mode, thereby further improving the binding of the target object to the solid surface.

It should be noted that the relative motion between the solid surface and the solution includes one of following: only the solid surface moves, only the solution moves and both the solid surface and the solution move. The above-mentioned “only the solid surface moves” means that the solution does not move but the solid surface moves. The above-mentioned “only the solution moves” means that the solid surface does not move but the solution moves.

In this embodiment, for a biomolecular interaction sensing experiment, the solid surface includes a sensor surface with ligands immobilized thereon; the target object includes analytes which are dissolved or dispersed in the solution; and a binding and a dissociation between the target object and the solid surface refer to a binding and a dissociation between the analytes and the ligands, respectively. The sensor includes a sensor at an optical fiber end-facet or a sensor at an optical cable end-facet, the end-facet is approximately perpendicular to a lightwave propagation direction in a part of optical fiber or optical cable adjacent to the sensor, and the choice of sensor at an optical fiber end-facet includes a surface plasmon resonance (SPR) sensor and the choice of sensor at an optical cable end-facet includes a bio-layer interferometry sensor. It should be noted that although this embodiment focuses on label-free biomolecular interaction experiments, the present disclosure has the same effect on labeled biomolecular interaction experiments. Therefore, the present disclosure is not limited to the examples listed herein. It should be noted that the above-mentioned “approximately perpendicular” includes substantially perpendicular or deviating from perpendicular by a certain angular range. For example, the angular range of the deviation may be between −2° and +2°, but is not limited to the angular range listed here.

The target object includes analytes in the form of large particles, and the analytes in the form of large particles may include one or more of metallic particles, exosome particles, cells, quantum dots and dielectric particles. Further, the velocity of the perpendicular relative movement between the solid surface (such as ligands on a sensor surface) and the solution is no less than 1/10 of the Brownian motion velocity of the target object in the solution, so as to increase the probability of collision between the solid surface and the target object (such as the analytes). Furthermore, when the velocity of the perpendicular relative movement between the sensor and the sample solution reaches or exceeds the Brownian motion velocity of the analyte, such a motion mode will increase the probability of ligand-analyte collision even more significantly. For the measurement of analyte particles with large masses and low Brownian motion velocities, such as metallic particles, exosome particles, cells, quantum dots and dielectric particles, the present disclosure can remarkably increase the velocity of relative movement between the analyte and the ligand. Of course, during the practical application, different velocities of the perpendicular relative movement may be selected according to different target objects (for example, target objects with different particle sizes), and the present disclosure is not limited to the examples listed herein.

This embodiment further provides a second mode of relative motion between a solid surface and a solution. In an interaction between the solid surface and a target object, the target object is dissolved or dispersed in the solution, and the solid surface and the solution make a relative motion, with the relative motion including a reciprocating movement parallel to the solid surface which is different from rotation around a center, in order to improve the binding and dissociation rates between the target object and the solid surface. The above-mentioned “rotation around a center” refers to such a movement in which, if we connect two arbitrary points on the solid surface to form a line segment a, and connect two arbitrary points in the solution to form a line segment b, the angle between line segments a and b changes, instead of having line segments a and b move relatively to each other with a constant angle between them.

For a biomolecular interaction sensing experiment, the solid surface includes a sensor surface with ligands immobilized thereon, the target object includes analytes which are dissolved or dispersed in the solution, and a binding and a dissociation between the target object and the solid surface refer to a binding and a dissociation between the analytes and the ligands, respectively. The sensor includes a sensor at an optical fiber end-facet or a sensor at an optical cable end-facet, the end-facet is approximately perpendicular to a lightwave propagation direction in a part of optical fiber or optical cable adjacent to the sensor, and the choice of sensor at an optical fiber end-facet includes a surface plasmon resonance (SPR) sensor and the choice of sensor at an optical cable end-facet includes a bio-layer interferometry sensor. In this embodiment, the sensor and sample solution conduct a reciprocating relative movement parallel to the sensor surface, which is different from rotation around a center. This mode of motion can also significantly improve the ligand-analyte binding efficiency compared to when there is no movement. In addition, the relative motion velocity under this motion mode does not vary with the spatial offset of the sensor from the center of the sample solution, so that the consistency between different testing results shall be improved compared with the rotating-around-a-center relative motion mode.

In an exemplary embodiment, a sensing apparatus is shown in FIG. 1. The sensing apparatus mainly includes a light source 10, a spectrometer 15, a directional coupler 11, and an optical fiber end-facet sensor 14. The sensor 14 is an surface plasmon resonance (SPR) sensor on the end-facet of an optical fiber, in which a surface plasmon resonance (SPR) resonant cavity is integrated on the end-facet of a 780 nm-wavelength single-mode fiber. The resonant cavity includes two nanoslit arrays with different periods on a 55 nm thick gold film. A central nanoslit array of the resonant cavity is 11×11 μm² in size, with a 645 nm nanoslit period. Another nanoslit array surrounds the central nanoslit array, which has a size of 100×100 μm² and a nanoslit period of 315 nm. The width of the nanoslits is 50 nm, and the depth of the nanoslits penetrates through the gold film. The sensing method of this embodiment is shown in FIG. 1. The broadband light emitted by light source 10, which is a super-luminescent diode here, couples into the single-mode fiber (SMF) 12. The directional coupler 11 routes the broadband light to the surface plasmon resonance (SPR) sensor 14 on the end-facet of the optical fiber. The optical fiber having a surface plasmon resonance (SPR) sensor 14 on the end-facet is vertically immersed into a sample solution 13, the analyte is dispersed or dissolved in the sample solution 13. The light reflects off the sensor back to the optical fiber and is then routed to a spectrometer 15 through a directional coupler 11, so that a reflection spectrum is measured. The surface plasmon resonance (SPR) effect produces a resonance dip near 850 nm wavelength in the reflection spectrum, whose central wavelength red-shifts as the amount of molecules adsorbed on the surface of the gold film increases. By measuring the central wavelength of the spectral resonance dip, the binding and dissociation of the analyte to the surface of the gold film (the sensor surface) will be detected.

FIGS. 4-6 show the mode of relative motion of the sensor and sample solution in the experiment. The sample solution is placed in a standard 96-well microtitre plate, with the sample solution in each well being 200 μL. An optical fiber having a surface plasmon resonance (SPR) sensor on the end-facet is vertically inserted into the wells and immersed into the sample solution, as shown in FIG. 1. The mode of relative motion is selected from the following four modes:

1) as shown in FIG. 6, the sensor does not move, while the microtitre plate conducts a circular rotation motion F, with a rotation diameter of about 1 mm and a rotation frequency of about 1000 rpm, which give a rotation speed of about 52 mm/s;

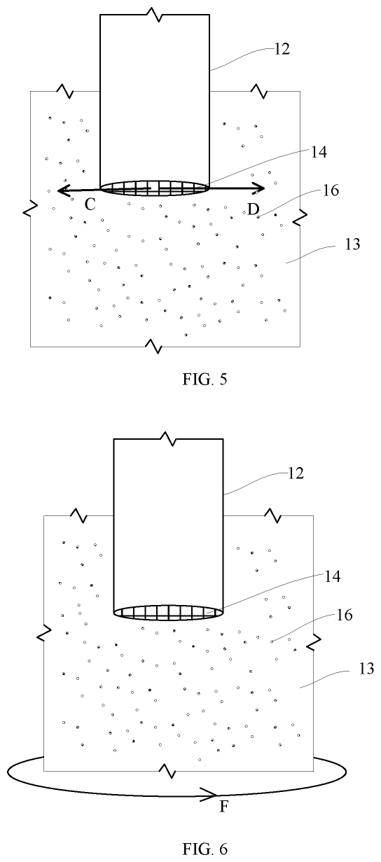

2) as shown in FIG. 5, the microtitre plate does not move, while the optical fiber having the surface plasmon resonance (SPR) sensor on the end-facet reciprocatively moves to the left and right along the horizontal direction (movements C and D), with a motion amplitude of 2 mm and a motion frequency of 3 Hz, which give an average motion velocity of 12 mm/s;

3) as shown in FIG. 4, the microtitre plate does not move, while the optical fiber having the surface plasmon resonance (SPR) sensor on the end-facet reciprocatively moves up and down along the vertical direction (movements A and B), with a motion amplitude of 2 mm and a motion frequency of 3 Hz, which give an average motion velocity of 12 mm/s;

4) the microtitre plate and the optical fiber having the surface plasmon resonance (SPR) sensor on the end-facet do not have a relative motion with respect to each other.

In this embodiment, two label-free biomolecular interaction experiments are used as examples to compare the experimental effects of the above motion modes. The experiments include 1) PPB-SA molecular interaction experiment; 2) BPA-hIgG molecular interaction experiment. Details are as follows:

1) PPB-SA Molecular Interaction Experiment:

The buffer solution used in this experiment is 4-(2-hydroxyethyl)-1-piperazineethanesulfonic acid (HEPES). The ligand on the sensor surface of the optical fiber end-facet SPR sensor is (poly-L-lysine)-(polyethylene glycol)-biotin (PPB) molecule. The sample solution (analyte solution) is streptavidin (SA) molecules in a HEPES solvent, with a concentration of 1 μg/mL.

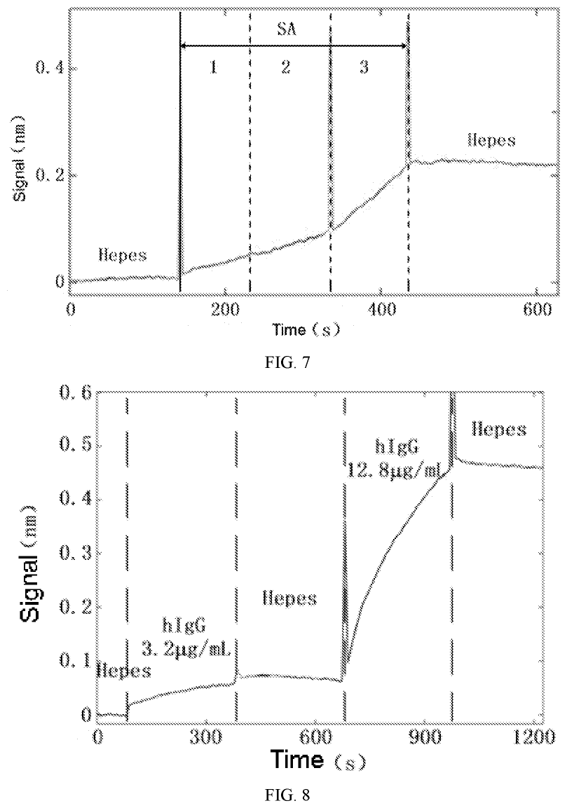

As shown in FIG. 7, the sensor is first immersed in the HEPES buffer solution, with the sensor and the sample solution unmoving. Then the sensor is immersed in the SA molecule solution and conducts three motion modes in sequence, including: 1) the sensor does not move, while the microtitre plate conducts a circular rotation motion F, with a rotation diameter of about 1 mm and a rotation frequency of about 1000 rpm, which give a rotation speed of about 52 mm/s; 2) the microtitre plate does not move, while the optical fiber having the surface plasmon resonance (SPR) sensor on the end-facet reciprocatively moves to the left and right along the horizontal direction (movements C and D), with a motion amplitude of 2 mm and a motion frequency of 3 Hz, which give an average motion velocity of 12 mm/s; 3) the microtitre plate does not move, while the optical fiber having the surface plasmon resonance (SPR) sensor on the end-facet reciprocatively moves up and down along the vertical direction (movements A and B), with a motion amplitude of 2 mm and a motion frequency of 3 Hz, which give an average motion velocity of 12 mm/s. At last, the sensor is immersed in the HEPES buffer solution, with the sensor and the sample solution unmoving. FIG. 7 shows the real-time binding and dissociation processes between the SA molecules and the sensor surface. The vertical axis is shift of the central wavelength of the resonance dip in the SPR reflection spectrum, which has a linear relationship with the amount of SA molecules adsorbed onto the sensor surface (the sharp peaks in FIG. 7 correspond to a quick transition of the sensor from one well to another, which do not always show up). As can be seen from FIG. 7, obviously, the reciprocating perpendicular motion of the optical fiber results in a faster binding rate of the SA molecules to the sensor surface. In addition, although the motion velocity of the circular rotation motion is much higher than that of the reciprocating horizontal motion, the binding rates of SA molecules to the sensor surface do not have much difference between these two cases.

2) BPA-hIgG Molecular Interaction Experiment:

The buffer solution used in this experiment is 4-(2-hydroxyethyl)-1-piperazineethanesulfonic acid (HEPES). The ligand on the sensor surface of the optical fiber end-facet SPR sensor is biotinylated protein A (BPA) molecule. The sample solution (analyte solution) is human immunoglobulin G (hIgG) molecules in a HEPES solvent.

As shown in FIGS. 8-10, the sensor is immersed in HEPES buffer solution, hIgG solution with a concentration of 3.2 μg/mL, HEPES buffer solution, hIgG solution with a concentration of 12.8 μg/mL and HEPES buffer solution in sequence. During the whole testing process, a same motion mode is used. FIGS. 8-10 respectively show the measurement results under the following three motion modes: 1) the microtitre plate and the optical fiber having the surface plasmon resonance (SPR) sensor on the end-facet do not have a relative motion with respect to each other; 2) the sensor does not move, while the microtitre plate conducts a circular rotation motion F, with a rotation diameter of about 1 mm and a rotation frequency of about 1000 rpm, which give a rotation speed of about 52 mm/s; and 3) the microtitre plate does not move, while the optical fiber having the surface plasmon resonance (SPR) sensor on the end-facet reciprocatively moves up and down along the vertical direction (movements A and B), with a motion amplitude of 2 mm and a motion frequency of 3 Hz, which give an average motion velocity of 12 mm/s. As can be seen from FIGS. 8-10, the binding rate of hIgG molecules to the sensor surface is remarkably increased when the motion mode changes from 1), 2) to 3). This increasing is even more significant for low concentration hIgG solutions.

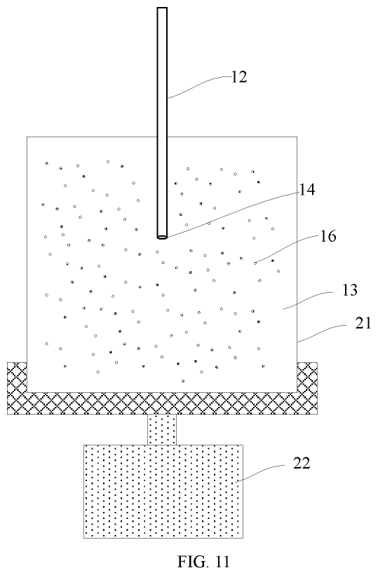

As shown in FIG. 11, this embodiment further provides a motion apparatus for a solid surface and a solution, including: a container 21, which holds a solution 13, with a target object 16 dissolved or dispersed in the solution 13; a solid surface, which includes a sensor surface with ligands immobilized thereon for measuring biomolecular interactions, the sensor 14 being immersed in the solution 13; and a drive motor 22, which is connected to the container 21 to make a reciprocating motion of the container, the reciprocating motion including a movement perpendicular to the sensor surface, in order to improve at least one of the following: binding rate, dissociation rate, binding uniformity, binding directionality, and binding density of the target object 16 to the sensor surface.

In this embodiment, the reciprocating motion of the container 21 driven by the drive motor 22 further includes a movement parallel to the surface of the sensor 14. The drive motor 22 drives the container 21 to move in directions perpendicular to the surface of the sensor 14 and in directions parallel to the surface of the sensor 14 at the same time.

As an example, the drive motor includes a voice coil motor. The voice coil motor drives a motion in a certain mode by using the interaction force between the magnetic poles of permanent magnets and/or electric current coils. It can realize linear motions and pendulum motions with finite angles. Compared with traditional linear motors, the voice coil motors have better high-frequency responses for driving high-speed reciprocating motions.

As an example, the container includes a microtitre plate or a centrifuge tube.

As an example, the sensor includes a sensor at an optical fiber end-facet or a sensor at an optical cable end-facet, the end-facet is approximately perpendicular to a lightwave propagation direction in a part of optical fiber or optical cable adjacent to the sensor, and the choice of sensor at an optical fiber end-facet includes a surface plasmon resonance (SPR) sensor and the choice of sensor at an optical cable end-facet includes a bio-layer interferometry sensor.

As described above, the modes of relative motion between a solid sensor surface and a sample solution and the related motion apparatuses of the present disclosure have the following beneficial effects:

1) The present disclosure provides a mode of relative motion in which the relative motion between the sensor surface and the solution is perpendicular to the solid surface. Compared with the traditional modes of relative motion in which the relative motion is parallel to a sensor surface, the mode of relative motion of the present disclosure can effectively improve the binding efficiency and dissociation efficiency between a ligand and an analyte given the same relative motion velocity between the sensor and the sample solution.

2) The perpendicular motion mode of the present disclosure will not cause significant nonuniformity of the flow rate of the sample solution, and it will effectively improve the binding uniformity, binding directionality and binding density of the analyte to the ligand.

3) The present disclosure can effectively improve the detection sensitivity for biomolecular interactions, and therefore shows promises for a broad range of applications in biosensing.

4) When the velocity of the perpendicular relative movement between the sensor and the sample solution is no less than 1/10 of the Brownian motion velocity of the analyte, such modes of relative motion will significantly increase the probability of ligand-analyte collision. For the measurement of analyte particles with large masses and low Brownian motion velocities, such as metallic particles, exosome particles, cells, quantum dots and dielectric particles, the present disclosure can remarkably increase the velocity of relative movement between the analyte and the ligand.

5) In the present disclosure, the sensor and sample solution conduct a reciprocating relative movement parallel to the sensor surface, which is different from rotation around a center. This mode of motion can also significantly improve the ligand-analyte binding efficiency compared to when there is no movement. In addition, the relative motion velocity under this motion mode does not vary with the spatial offset of the sensor from the center of the sample solution, so that the consistency between different testing results shall be improved compared with the rotating-around-a-center relative motion mode.

Therefore, the present disclosure effectively overcomes various shortcomings in the traditional technology and has high industrial utilization value.

The above-described embodiments are merely illustrative of the principles of the disclosure and its effects, and are not intended to limit the disclosure. Modifications or variations of the above-described embodiments may be made by those skilled in the art without departing from the spirit and scope of the disclosure. Therefore, all equivalent modifications or changes made by those who have common knowledge in the art without departing from the spirit and technical concept disclosed by the present disclosure shall be still covered by the claims of the present disclosure.

1. A mode of relative motion between a solid surface and a solution, wherein in an interaction between the solid surface and a target object, the target object is dissolved or dispersed in the solution, and the solid surface and the solution make a relative motion, with the relative motion including a relative movement perpendicular to the solid surface, in order to improve at least one of the following: binding rate, dissociation rate, binding uniformity, binding directionality and binding density of the target object to the solid surface.

2. The mode of relative motion between a solid surface and a solution according to claim 1, wherein in binding and dissociation processes between the solid surface and the target object, the relative motion between the solid surface and the solution includes a unidirectional opposite movement perpendicular to the solid surface, in order to improve at least one of the following: binding rate, dissociation rate, binding uniformity, binding directionality, and binding density of the target object to the solid surface.

3. The mode of relative motion between a solid surface and a solution according to claim 1, wherein in binding and dissociation processes between the solid surface and the target object, the relative motion between the solid surface and the solution includes reciprocating forward and backward movements perpendicular to the solid surface, in order to improve at least one of the following: binding rate, dissociation rate, binding uniformity, binding directionality, and binding density of the target object to the solid surface.

4. The mode of relative motion between a solid surface and a solution according to claim 1, wherein the relative motion includes a combination of a relative movement perpendicular to the solid surface and a relative movement parallel to the solid surface.

5. The mode of relative motion between a solid surface and a solution according to claim 4, wherein the relative movement perpendicular to the solid surface and the relative movement parallel to the solid surface are simultaneously made.

6. The mode of relative motion between a solid surface and a solution according to claim 4, wherein the relative movement parallel to the solid surface includes one of the following: a unidirectional movement, a bidirectional reciprocating movement and a rotating movement.

7. The mode of relative motion between a solid surface and a solution according to claim 1, wherein the relative motion between the solid surface and the solution includes one of following: only the solid surface moves, only the solution moves and both the solid surface and the solution move.

8. The mode of relative motion between a solid surface and a solution according to claim 1, wherein a velocity of the perpendicular relative movement between the solid surface and the solution is not less than 1/10 of the Brownian motion velocity of the target object in the solution, in order to increase the probability of collision between the solid surface and the target object.

9. The mode of relative motion between a solid surface and a solution according to claim 8, wherein the target object includes analytes in the form of large particles, which are selected from but are not limited to a group consisting of metallic particles, exosome particles, cells, quantum dots and dielectric particles, and a combination thereof.

10. The mode of relative motion between a solid surface and a solution according to claim 1, wherein, for a biomolecular interaction experiment, the solid surface comprises a sensor surface with ligands immobilized thereon, and the target object comprises analytes which are dissolved or dispersed in the solution; a binding and a dissociation between the target object and the solid surface refer to a binding and a dissociation between the analytes and the ligands, respectively.

11. The mode of relative motion between a solid surface and a solution according to claim 10, wherein the sensor comprises a sensor at an optical fiber end-facet or a sensor at an optical cable end-facet, wherein the end-facet is approximately perpendicular to a lightwave propagation direction in a part of optical fiber or optical cable adjacent to the sensor, and the choice of sensor at an optical fiber end-facet includes a surface plasmon resonance (SPR) sensor and the choice of sensor at an optical cable end-facet includes a bio-layer interferometry sensor.

12. A mode of relative motion between a solid surface and a solution, wherein in an interaction between the solid surface and a target object, the target object is dissolved or dispersed in the solution, and the solid surface and the solution make a relative motion, with the relative motion including a reciprocating movement parallel to the solid surface other than rotation around a center, in order to improve the binding and dissociation rates between the target object and the solid surface.

13. The mode of relative motion between a solid surface and a solution according to claim 12, wherein, for a biomolecular interaction experiment, the solid surface comprises a sensor surface with ligands immobilized thereon, and the target object comprises analytes which are dissolved or dispersed in the solution; a binding and a dissociation between the target object and the solid surface refer to a binding and a dissociation between the analytes and the ligands, respectively; the sensor comprises a sensor at an optical fiber end-facet or a sensor at an optical cable end-facet, wherein the end-facet is approximately perpendicular to a lightwave propagation direction in a part of optical fiber or optical cable adjacent to the sensor, and the choice of sensor at an optical fiber end-facet includes a surface plasmon resonance (SPR) sensor and the choice of sensor at an optical cable end-facet includes a bio-layer interferometry sensor.

14. A motion apparatus for a solid surface and a solution, comprising:
a container, which holds a solution, with a target object dissolved or dispersed in the solution;
a solid surface, which comprises a sensor surface with ligands immobilized thereon for measuring biomolecular interactions, the sensor being immersed in the solution;
and a drive motor, which is connected to the container to make a reciprocating motion of the container, the reciprocating motion including a movement perpendicular to the sensor surface, in order to improve at least one of the following: binding rate, dissociation rate, binding uniformity, binding directionality, and binding density of the target object to the sensor surface.

15. The motion apparatus for a solid surface and a solution according to claim 14, wherein the reciprocating motion of the container driven by the drive motor further comprises a movement parallel to the sensor surface, so that the perpendicular and parallel movements of the container are simultaneously driven.

16. The motion apparatus for a solid surface and a solution according to claim 14 or 15, wherein the drive motor comprises a voice coil motor.

17. The motion apparatus for a solid surface and a solution according to claim 14, wherein the container comprises a microtitre plate or a centrifuge tube.

18. The motion apparatus for a solid surface and a solution according to claim 14, wherein the sensor comprises a sensor at an optical fiber end-facet or a sensor at an optical cable end-facet, the end-facet is approximately perpendicular to a lightwave propagation direction in a part of optical fiber or optical cable adjacent to the sensor, and the choice of sensor at an optical fiber end-facet includes a surface plasmon resonance (SPR) sensor and the choice of sensor at an optical cable end-facet includes a bio-layer interferometry sensor.

19. The motion apparatus fora solid surface and a solution according to claim 15, wherein the drive motor comprises a voice coil motor.
